(19) United States
(12) Patent Application Publication
(10) Pub. No.: US 2020/0009281 A1
(43) Pub. Date: Jan. 9, 2020

(54) ODOR CONTROL AGENT

(71) Applicant: Dow Global Technologies LLC, Midland, MI (US)

(72) Inventors: Stephen E. Fosdick, Pearland, TX (US); Sung-Yu Ku, Pearland, TX (US); Ashwin R. Bharadwaj, Pearland, TX (US)

(21) Appl. No.: 16/495,159

(22) PCT Filed: Mar. 21, 2018
(86) PCT No.: PCT/US2018/023452

§ 371 Date: Sep. 18, 2019

Related U.S. Application Data
(60) Provisional application No. 62/474,663, filed on Mar. 22, 2017.

(51) Int. Cl. A61L 9/013; A01K 1/015; A61K 31/194; A61K 31/198; A61K 31/785; A61K 31/717; A61K 33/04; A61K 33/42; A61K 33/06

(52) U.S. Cl. CPC A61L 9/013; A01K 1/0155; A61K 31/194; A61K 31/198; A61K 2300/00; A61K 31/717; A61K 33/04; A61K 33/42; A61K 33/06; A61K 31/785

(57) ABSTRACT

An odor control agent comprising: a filler; a binder; an active comprising an aminopolycarboxylic acid and a polyprotic acid. An odor control agent comprising: an active comprising an aminopolycarboxylic acid and a polyprotic acid, wherein at least one of the aminopolycarboxylic acid or the polyprotic acid is the salt form of the acid.

### BACKGROUND

Animal litter is used as a catch material for feces and urine. These biological by-products can develop strong odors due to evolution of malodorous compounds, especially ammonia. Ammonia results from microbial action on urea and/or uric acid in the animal waste.

The odor from animal litters is distressing to humans and at sufficiently high levels, may be toxic. In addition, the odor is distressing to the welfare of the animals, especially in closed environments such as poultry houses and horse stalls/barns. Moreover, emissions from animal litter may contribute to the greenhouse effect. It is desirable, therefore, to find ways of controlling odors from animal litter.

### STATEMENT OF INVENTION

An odor control agent comprising: a filler; a binder; an active comprising an aminopolycarboxylic acid and a polyprotic acid.

An odor control agent comprising: an active comprising a aminopolycarboxylic acid and a polyprotic acid, wherein at least one of the aminopolycarboxylic acid or the polyprotic acid is the salt form of the acid.

### DETAILED DESCRIPTION

Unless otherwise indicated, numeric ranges, for instance as in “from 2 to 10,” are inclusive of the numbers defining the range (e.g., 2 and 10).

Unless otherwise indicated, ratios, percentages, parts, and the like are by weight.

The terms “prevent” or “suppress” as used herein means at least partly reducing the amount of ammonia that would otherwise be formed in animal waste in the absence of the additive and is reported as a “percent improvement”. In some embodiments, the percent improvement is at least 50 percent, alternatively at least 70 percent, alternatively at least 90 percent, or alternatively 100 percent, as measured for instance by colorimetric indicators (e.g., using Drager tubes as shown in the Examples), optical transmission absorption methods and/or gas chromatography in real time animal usage or laboratory testing method that mimics urine degradation. The percent improvement is relative a system containing the same carrier material and bacteria as the test environment but omitting the active.

The term “animal” as used in this specification generally means non-human animals. Non-limiting examples include domesticated animals, zoo animals, farm animals, pets, and other animals that spend some of their time in a partially or fully enclosed environment. More specific examples include, without limitation, cats, dogs, poultry (e.g., chickens), horses, cows, swine, rabbits, goats, and rodents (e.g., guinea pigs, hamsters, ferrets, mice, and rats).

The term “waste” refers to any animal waste product that may be transformed by bacteria into ammonia. In one instance, waste refers to any animal waste product that contains urea, uric acid, or both. Examples include animal urine and excrement (feces, droppings).

The present disclosure describes an odor control agent which works to suppress ammonia formation. In one instance, the odor control agent described herein comprises an active. In another instance, the odor control agent described herein comprises a filler, a binder and an active. In some instances, the odor control agent is a component of an odor control system which further comprises a carrier.

The filler is a low-density material to which the active is joined using the binder. In one instance, the filler is a mineral, for example, mica, zeolite, or vermiculite. Foamed polymeric materials are an example of a suitable filler. In some instances, the low-density material may be polystyrene, polyethylene, polypropylene, or polyethylene terephthalate. The filler may be a biodegradable polymer, for example modified starches, polyvinyl alcohol, polybutylene succinate, polylactic acid, or cellulose esters. The filler is selected such that it is safe for use in proximity to animals. As used herein, “low-density” means that the filler is low-density as compared to the density of the carrier material. In one instance, the filler density is 5 to 95 percent the density of the carrier material. In one instance, the filler density is 5 to 85 percent the density of the carrier material. In one instance, the filler density is 10 to 75 percent the density of the carrier material. In one instance, the filler density is 10 to 65 percent the density of the carrier material. In one instance, the filler density is 15 to 55 percent the density of the carrier material. In one instance, the filler density is 15 to 45 percent the density of the carrier material. In one instance, the filler density is 15 to 40 percent the density of the carrier material. In one instance, the filler density is 5 to 40 percent the density of the carrier material. In one instance, the filler density is 20 to 50 percent the density of the carrier material. In one instance, the filler density is 20 to 45 percent the density of the carrier material. In one instance, the filler density is 20 to 40 percent the density of the carrier material. In one instance, the filler density is 10 to 95 percent the density of the carrier material. In one instance, the filler density is 20 to 95 percent the density of the carrier material. In one instance, the filler density is 30 to 95 percent the density of the carrier material. In one instance, the filler density is 40 to 95 percent the density of the carrier material. In one instance, the filler density is 50 to 95 percent the density of the carrier material. In one instance, the filler density is 60 to 95 percent the density of the carrier material. In one instance, the filler density is 70 to 95 percent the density of the carrier material.

In some embodiments, the binder is a liquid solution which, when combined with the active and the filler, forms granules such that during transport and typical use, a majority of the granules will substantially hold their shape. In some instances the binder is water, salt solutions, solutions of EDTA salts, glycols, propylene glycol, glycerin, polyethylene glycol, polypropylene glycol, polyalkylene glycol, lubricants, or a combination thereof. Examples of suitable lubricants are the UCON lubricants available from The Dow Chemical Company, for example UCON OSP 32, UCON OSP 46, UCON OSP 68, UCON OSP 150, and UCON OSP 220. For example, a suitable polyethylene glycol is Carbowax, available from The Dow Chemical Company. In some embodiments, the binder is a clay or mineral-based surfactant, based on bentonite, hectorite, or aluminosilicate. Preferably, the binder is a liquid or emulsion at room temperature. Preferably, the liquid binder has low toxicity.

The active comprises an aminopolycarboxylic acid and a polyprotic acid. Preferably, at least one of the aminopolycarboxylic acid and the polyprotic acids are free acids and are not the salt form of the acids. In one instance, the active is prepared by combining the free acid form of a aminopolycarboxylic acid and the salt form of a polyprotic acid. In one instance, the active is prepared by combining the salt form of a aminopolycarboxylic acid and the free acid form of a polyprotic acid. It is understood that equilibrium chemistry will occur between the free acid and the salt form of the acid of the active which may serve to partially neutralize a portion of the free acid. The aminopolycarboxylic acid functions by preventing formation of ammonia, an odor causing compound. In some embodiments, the aminopolycarboxylic acid has an ethylenediamine or diethylenetriamine backbone. In some embodiments, the aminopolycarboxylic acid is ethylenediaminetetraacetic acid, diethylenetriaminepentaacetic acid, N-hydroxyethylethylenediaminetriacetic acid, or a mixture of two or more thereof.

The polyprotic acid used herein comprises an acid which neutralizes or suppresses the formation of ammonia and is safe for use with animals. The polyprotic acid is preferably a free acid, meaning an acid that has not been deprotonated. The polyprotic acid is preferably not the salt-form of the acid. A polyprotic acid is an acid which is capable of losing more than one proton per molecule. In one instance, the polyprotic acid used herein is citric acid or citrate. Other polyprotic acids include sulfuric, sulfurous, phosphoric, carbonic, malic, terephthalic, tartaric, oxalic, malonic, phthalic, and aspartic. It is understood that the polyprotic acid will be present in equilibrium, and a portion of the acid will be present in the free acid form. In one instance, at least 1% of the polyprotic acid is present in the free acid form. In one instance, at least 5% of the polyprotic acid is present in the free acid form. In one instance, at least 10% of the polyprotic acid is present in the free acid form. In one instance, at least 15% of the polyprotic acid is present in the free acid form. In one instance, at least 20% of the polyprotic acid is present in the free acid form. In one instance, at least 25% of the polyprotic acid is present in the free acid form. In one instance, at least 50% of the polyprotic acid is present in the free acid form. In one instance, at least 60% of the polyprotic acid is present in the free acid form. In one instance, at least 70% of the polyprotic acid is present in the free acid form. In one instance, at least 80% of the polyprotic acid is present in the free acid form. In one instance, at least 90% of the polyprotic acid is present in the free acid form. In one instance, at least 99% of the polyprotic acid is present in the free acid form.

In one instance, the binder comprises 0.01 to 50 percent of the composition of the odor control agent. In one instance, the aminopolycarboxylic acid comprises 0.1 to 99 percent of the composition of the odor control agent. In one instance, the polyprotic acid comprises 0.1 to 99 percent of the composition of the odor control agent preferably between 0.1 and 50 percent. In one instance, the filler comprises 1 to 90 percent of the composition of the odor control agent. In one instance, the filler comprises between 1 and 50 percent of the composition of the odor control agent.

The carrier material may be any material that is typically used as a bedding or absorbent for animals and their waste and includes, for instance, swellable clays (e.g., bentonite and montmorillonite), non-swellable clays (e.g., kaolin), wood shavings, hay, wood chips, pelletized saw dust, paper, chopped corn cobs, peanut hulls, wood pulp, wheat grass, or mixtures thereof. In some embodiments, the carrier material is bentonite clay. In some embodiments, the animal is a cat and the carrier material is bentonite clay. In some embodiments, the animal is a cat and the carrier material is wood chips, wood shavings, paper byproducts, pine pellets, ground corn cob or pelletized saw dust. In some embodiments, the animal is poultry and the carrier material is wood shavings. In some embodiments, the animal is a horse and the carrier material is wood chips, wood shavings, or pelletized saw dust.

As described above, an odor control system comprises the odor control agent and the carrier material as described herein. The quantity of odor control agent added to the carrier material is sufficient to suppress ammonia as described herein. The quantity of odor control agent added to the carrier material is sufficient to provide odor control.

In another aspect, the invention provides improved efficiency in prevention of urea degradation due to the preferred placement of the odor control agent at or near the surface of the litter particles.

The compositions described herein may contain other additives, besides the odor control agent and carrier material, that are typically used in animal litters. These include, but are not limited to fillers, humectants, disintegrants, odor absorbing materials (e.g., sodium carbonate, potassium carbonate, siliceous material, opaline silica, activated carbon, sodium bisulfate complex, or corn starch), zeolite, dedusting agents (e.g., gaur gum, PTFE coated clay, or fluoropolymers), antimicrobials such as bronopol and silver based compounds, fragrances, other chelants (diethylenetriaminepentaacetic acid (DTPA) for example), gypsum, small molecule organic acids, polymers with neutralization capacity or acid groups (e.g., cellulose acetate, polyxcarboxylates), rice flour, quaternary amines, probiotic bacteria and/or ammonia oxidizing bacteria.

In some embodiments, the carrier material described herein is free of other odor preventing additives, for instance it is free of one or more of: an alkali metal tetraborate n-hydrate, alum, other transition metal salts (e.g., Zn, copper salts) and/or boron compounds.

The addition of the odor control agent to the carrier material, as described herein, results in the prevention of ammonia, thereby significantly reducing undesirable animal odors. The odor control agent can be incorporated with the carrier material by a variety of standard techniques known to those skilled in the art including, for instance, solids mixing (including dry blending or co-grinding), spreading, sprinkling, and the like.

For instance, in one embodiment, the odor control agent may be sprinkled over the top of a bed of the carrier material. In this embodiment the bed of carrier material may be further agitated to mix the odor control agent deeper into the material.

In another embodiment, the odor control agent may be dry blended with the carrier material and packaged together prior to use as an animal litter. In a preferred embodiment, the odor control agent is made into granules prior to dry blending such that the granules and the carrier material are of a similar size and shape so to inhibit demixing or stratification of the odor control agent from the carrier material.

In any of the embodiments above, different equipment may be used in order to accomplish the incorporation of the odor control agent and the carrier material.

When mixing the odor control agent, it may be desirable to match the particle density as well as shape of the carrier material to control particle segregation.

Other mixing techniques may include dry briquetting the mixture of carrier material and odor control agent to have both incorporated uniformly in the process (e.g., a size range from hundreds of microns to millimeters may be suitable).

In some embodiments, the odor control agent may be applied to the carrier material prior to the animal releasing its waste on the carrier material. In some embodiments, the odor control agent may be applied or reapplied to the carrier material for second or subsequent generations of use.

A person of ordinary skill in the art can readily determine the effective amount of odor control agent used in combination with the carrier material. By way of non-limiting example, suitable amounts may include at least 0.01 weight percent, preferably at least 0.1 weight percent, more preferably at least 0.15 weight percent, based on the total weight of the carrier material. Although there is no particular upper limit on the amount of the odor control agent, in some embodiments it may be desirable to use 10 weight percent or less, alternatively 8 weight percent or less, alternatively 5 weight percent or less, alternatively 1.2 weight percent or less, or alternatively 0.5 weight percent or less, based on the total weight of the carrier material.

The addition of the odor control agent to a carrier material reduces undesirable animal odors. The odor control agent can be incorporated with the carrier material by a variety of standard techniques known to those skilled in the art including, for instance, solids mixing (including dry blending or co-grinding), spreading, sprinkling, and the like.

The active is prepared by spraying the filler with a binder and then blending the filler with the aminopolycarboxylic acid and a polyprotic acid so that the dry particles are attached to the filler by the binder at a desired concentration. Alternatively, the active is prepared by dissolving or suspending the aminopolycarboxylic acid and the polyprotic acid in a binder solution followed by coating of the filler with the mixture. The active is optionally dried to evaporate water or a solvent component that may optionally be present in the binder. Preferably, the binder is at least partially water soluble.

Some embodiments of the invention will now be described in detail in the following Examples.

### Examples

Synthetic Urine Preparation.

In these examples, synthetic urine is prepared as follows. In a bottle add the following components: Urea (ACS Reagent Grade): 18.8 g, and 250 g of deionized water. The bottle is capped and mixed to dissolve urea. The pH of the solution is measured to confirm the pH is between 6.5 and 7.

Preparation of Innoculant.

In these examples, inoculant is prepared as follows.

Immediately before use, mix 6.25 mL of Urease Enzyme from Canavalia ensiformis (Jack bean) in Glycerol (Type III, 500-800 units/mL) with the full volume of Synthetic Urine prepared above.

Active Mixture Preparation.

In Examples 1 through 4 the indicated active components were mixed directly with the absorbent according to the amounts indicated in Table 2. The general procedure for Examples 5 through 9 is to charge polystyrene foam beads (diameter approximately 2-7 mm) into a 1 gallon plastic container, spray-coat the beads with a liquid solution of K₂EDTA in water, sprinkling solid citric acid on the beads, and drying in a low temperature oven (50° C.). Quantities for Examples 5 through 9 are shown in Table 1.

TABLE 1

| | Weight of | Preparation | Preparation | Preparation |
|---|---|---|---|---|
| | Polystyrene | Concentration | Weight | Weight |
| Exam- | Foam | of K₂EDTA | K₂EDTA | Citric |
| ple | Beads (g) | Solution (Wt %) | solution (g) | Acid (g) |
| 5 | 20.0 | 40 | 58.0 | 0.0 |
| 6 | 5.0 | 4 | 1.4 | 10.9 |
| 7 | 5.0 | 4 | 5.5 | 10.5 |
| 8 | 20.0 | 40 | 58.0 | 23.0 |
| 9 | 5.0 | 40 | 26.2 | 0.5 |

Sample Preparation.

Charge a plastic bag with 100 g of untreated NATIONAL™ 12 Bentonite (particle size approximately 1.6 mm) cat litter. Add the active component or mixture, as prepared in the Active Mixture Preparation section, to the bag containing the untreated litter. Seal the bag to prevent loss of material and mix by shaking for 30 seconds. Pour the mixture into a hexagonal plastic weighboat contained within a 1 gallon sealable plastic container having a lid with a hole covered with tape. Level the litter in each test sample by tapping on the container. Prepare the sample inoculant as above. Pour 10 mL of inoculant into the weighboat containing litter and treatment.

Headspace Ammonia Measurement.

The ammonia in the headspace of each test container, prepared according to the Sample Preparation section, is sampled using a SENSIDYNE® AP-20S Aspirating Detector Tube Pump. Low (0.2-20 ppm) or high (5-260 ppm) range SENSIDYNE® Ammonia Gas Detector Tubes are used to quantify the headspace ammonia concentration. The following procedure is used for ammonia detection and quantification in each test sample. 1. Break both ends of the detector tube using the breaking port on pump. 2. Point the arrow mark on the detector tube towards the aspirating pump. Insert the detector tube securely into the rubber tube connector of the aspirating pump. 3. To sample the test headspace, remove small strip of tape sealing the lid of the sealable plastic container. Insert the detector tube (attached to the aspirating pump) into the sampling hole to the specified measurement distance. Insert the tube into the headspace to 45 mm, equal to the thick blue line on the bottom end of the gas detector tube). 4. Hold the pump at the 45 mm measurement distance. Pull the pump handle at full stroke to the locked position. Wait for 1 minute until sampling is complete which is confirmed with the flow indicator of the pump. The instruction manual of the aspirating pump will give more details if necessary. 5. Read the scale at the maximum point of the stained layer (yellow in color). Read and report the concentration immediately after measurement. If the reading is off scale, for example 20 ppm for the low range tubes, repeat the measurement using the high range gas detector tube. 6. After sampling seal the sampling hole lid of the sealable plastic container with tape 7 Ammonia samples are collected after 1 day with results reported in Table 2.

The results are summarized in the following table.

TABLE 2

| | | | Active Treatment | | |
|---|---|---|---|---|---|
| | | | Level | | |
| Ex- | | | | ppm | ppm |
| am- | | | ppm | Citric | Ammo- |
| ple | Absorbent | Active | K₂EDTA | Acid | nia |
| 1 | NATIONAL ™ | None | 0.00E+00 | 0.00E+00 | 160 |
| | 12 Bentonite | | | | |
| 2 | NATIONAL ™ | K₂EDTA | 1.00E+04 | 0.00E+00 | 190 |
| | 12 Bentonite | | | | |
| 3 | NATIONAL ™ | Citric Acid | 0.00E+00 | 1.00E+04 | 140 |
| | 12 Bentonite | | | | |
| 4 | NATIONAL ™ | K₂EDTA + Citric | 1.00E+04 | 1.00E+04 | 170 |
| | 12 Bentonite | Acid | | | |
| 5 | NATIONAL ™ | K₂EDTA treated | 9.19E+03 | 0.00E+00 | 260 |
| | 12 Bentonite | beads | | | |
| 6 | NATIONAL ™ | K₂EDTA + Citric | 8.25E+01 | 1.64E+04 | 35 |
| | 12 Bentonite | Acid treated | | | |
| | | beads | | | |
| 7 | NATIONAL ™ | K₂EDTA + Citric | 8.25E+02 | 1.57E+04 | 45 |
| | 12 Bentonite | Acid treated | | | |
| | | beads | | | |
| 8 | NATIONAL ™ | K₂EDTA + Citric | 8.53E+03 | 8.20E+03 | 105 |
| | 12 Bentonite | Acid treated | | | |
| | | beads | | | |
| 9 | NATIONAL ™ | K₂EDTA + Citric | 1.57E+04 | 8.25E+02 | 260 |
| | 12 Bentonite | Acid treated | | | |
| | | beads | | | |

1. An odor control agent comprising:
a filler;
a binder;
an active comprising an aminopolycarboxylic acid and a polyprotic acid.

2. The odor control agent of claim 1, wherein the filler comprises a material such as foamed polystyrene, mineral, or cellulosic material.

3. The odor control agent of claim 1, wherein the binder comprises a polyalkylene glycol derivative, a surfactant, or water.

4. The odor control agent of claim 1, wherein the polyprotic acid is selected from one or more of citric acid, citrate, sulfuric acid, sulfurous acid, phosphoric acid, carbonic acid, malic acid, terephthalic acid, tartaric acid, oxalic acid, malonic acid, phthalic acid, and aspartic acid.

5. The odor control agent of claim 1, wherein the aminopolycarboxylic acid comprises an acid having an ethylenediamine or diethylenetriamine backbone.

6. The odor control agent of claim 1, wherein the aminopolycarboxylic acid comprises ethylenediaminetetraacetic acid, diethylenetriaminepentaacetic acid, N-hydroxyethylethylenediaminetriacetic acid, or a mixture of two or more thereof.

7. An odor control system comprising the odor control agent of claim 1 and a carrier.

8. The odor control system of claim 7, wherein the carrier comprises clay, wood shavings, hay, wood chips, pelletized saw dust, paper, chopped corn cobs, peanut hulls, wood pulp, wheat grass, or mixtures thereof.

9. An odor control agent comprising:
an active comprising an aminopolycarboxylic acid and a polyprotic acid, wherein at least one of the aminopolycarboxylic acid or the polyprotic acid is the salt form of the acid.

10. The odor control agent of claim 9, wherein the polyprotic acid is selected from one or more of citric acid, citrate, sulfuric acid, sulfurous acid, phosphoric acid, carbonic acid, malic acid, terephthalic acid, tartaric acid, oxalic acid, malonic acid, phthalic acid, and aspartic acid.

11. The odor control agent of claim 9, further comprising a filler.

12. The odor control agent of claim 9, further comprising a binder.

13. The odor control agent of claim 9, wherein the active is prepared by combining the salt form of an aminopolycarboxylic acid and the free form of a polyprotic acid.
